(19) United States
(12) Patent Application Publication
(10) Pub. No.: US 2007/0067041 A1
(43) Pub. Date: Mar. 22, 2007

(54) Inflatable facial implant and associated method

(76) Inventor: Thomas G. Kotoske, Paradise Valley, AZ (US)

Correspondence Address: BARNES & THORNBURG LLP, 11 SOUTH MERIDIAN, INDIANAPOLIS, IN, 46204, US

(21) Appl. No.: 11/229,218

(22) Filed: Sep. 16, 2005

(51) Int. Cl. A61F 2/02

(52) U.S. Cl. 623/17.18

(57) ABSTRACT

An inflatable facial implant includes a base having a first side and a second side. A bladder wall is secured to the first side of the base. The bladder wall is more flexible than the base. Surgical methods of using such an implant are also disclosed.

### FIELD OF THE DISCLOSURE

The present disclosure relates generally to facial implants and methods of using the same.

### BACKGROUND

During the lifetime of a patient, the patient may require or elect to have a cosmetic or reconstructive facial procedure. One such type of procedure is facial augmentation. Traditional facial augmentation has included the use of implants such as fat transfers, alloplastic implants, synthetic solid implants, and/or injectables (e.g., collagen or silicone injections).

Subsequent to implantation, there is occasionally the need to adjust or correct the implant. For example, some of these implants cannot be corrected beyond, for example, two years subsequent to implantation. As such, the patient is forced to either not enjoy the benefits of the previous procedure or elect for a revision procedure.

### SUMMARY

According to one aspect of the present disclosure, an inflatable facial implant includes a base having a first side and a second side. A bladder wall is secured to the first side of the base. The bladder wall is more flexible than the base.

At least a portion of the second side of the base may be concave. The second side of the base may be contoured to substantially conform to a subcutaneous anatomical structure of a human such as soft tissue or bone. In some embodiments, the second side of the base is contoured to substantially conform to a cheekbone or mandible of a human.

The implant may be configured as a malar implant, chin implant, nasolabial fold implant, meliolabial fold implant, glabellar fold implant, lip augmentation implant, nasal implant, or any other type of facial implant.

In certain embodiments, the implant may include a fill port positioned in the bladder wall. The fill port may be arranged substantially flush-mount with a contoured outer surface of the implant.

The bladder wall may be constructed of a self-sealing polymer.

According to another aspect of the disclosure, an inflatable facial implant includes a base having a first side and a second side. The second side of the base is contoured to substantially conform to a subcutaneous anatomical structure of a human face. A bladder wall is secured to the first side of the base.

In certain embodiments, the bladder wall is more flexible than the base.

At least a portion of the second side of the base may be concave.

The second side of the base may be contoured to substantially conform to soft tissue or bone. In some embodiments, the second side of the base is contoured to substantially conform to a cheekbone or mandible of a human.

The implant may be configured as a malar implant, chin implant, nasolabial fold implant, meliolabial fold implant, glabellar fold implant, lip augmentation implant, nasal implant, or any other type of facial implant.

In certain embodiments, the implant may include a fill port positioned in the bladder wall. The fill port may be arranged substantially flush-mount with a contoured outer surface of the implant.

The bladder wall may be constructed of a self-sealing polymer.

According to another aspect of the disclosure, a reconstructive or cosmetic surgical method includes the step of injecting an initial quantity of fluid into a bladder of a facial implant. The bladder of the facial implant is secured to a base which is less flexible than the bladder. The facial implant is then implanted into a patient such that a base of the facial implant contacts a subcutaneous tissue. Subsequent to implantation, an additional quantity of fluid may be percutaneously injected into the bladder of the facial implant.

In certain embodiments, the bladder may be constructed of a self-sealing polymer. A needle may be used to percutaneously pierce the bladder to add or remove fluid therefrom.

In certain embodiments, the facial implant may include a subcutaneous port having a self-sealing septum. A needle may be used to percutaneously pierce the septum to add or remove fluid from the bladder.

The implant may be configured as a malar implant, chin implant, nasolabial fold implant, meliolabial fold implant, glabellar fold implant, lip augmentation implant, nasal implant, or any other type of facial implant.

The above and other features of the present disclosure will become apparent from the following description and the attached drawings.

### BRIEF DESCRIPTION OF THE DRAWINGS

The detailed description particularly refers to the accompanying figures in which:

FIG. 1 is a perspective view of an inflatable malar implant;

FIG. 2 is a plan view of the malar implant of FIG. 1;

FIG. 3 is a fragmentary cross sectional view showing the malar implant of FIG. 1 subsequent to being implanted into a patient;

FIG. 4 is a perspective view of an inflatable chin implant;

FIG. 5 is a plan view of the chin implant of FIG. 4;

FIG. 6 is a fragmentary cross sectional view showing the chin implant of FIG. 4 subsequent to being implanted into a patient;

FIG. 7 is a perspective view of an inflatable nasolabial fold implant;

FIG. 8 is a plan view of the nasolabial fold implant of FIG. 7;

FIG. 9 is a fragmentary cross sectional view showing the nasolabial fold implant of FIG. 7 subsequent to being implanted into a patient; and

FIGS. 10 and 11 are views similar to FIG. 3, but showing a needle being used to add fluid to, or remove fluid from, the bladder of the malar implant.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the disclosure to the particular forms disclosed, but on the contrary, the intention is to cover all modifications, equivalents, and alternatives following within the spirit and scope of the invention as defined by the appended claims.

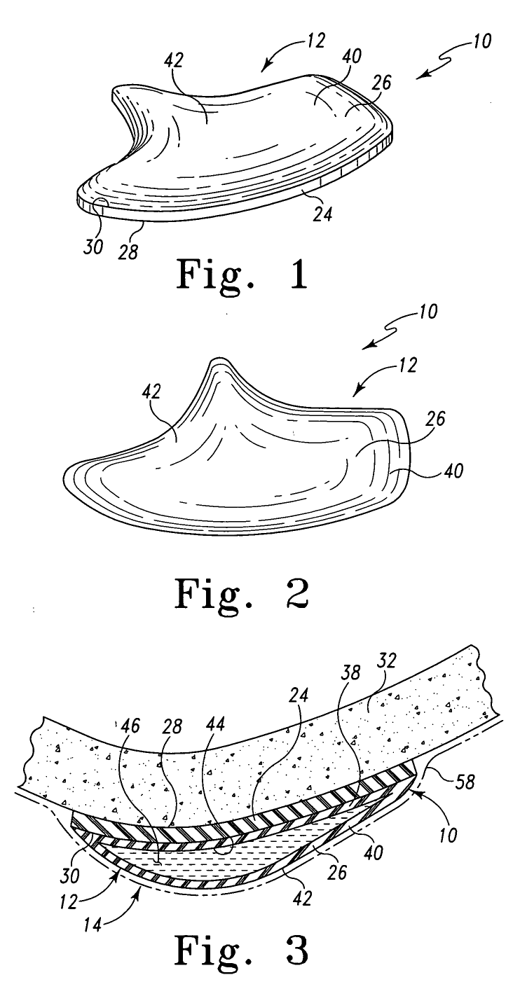

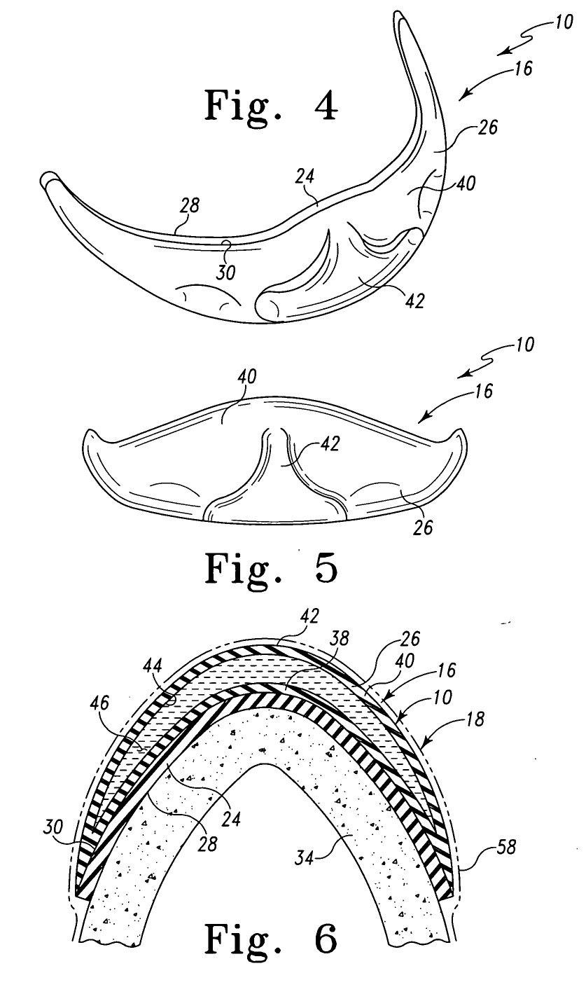

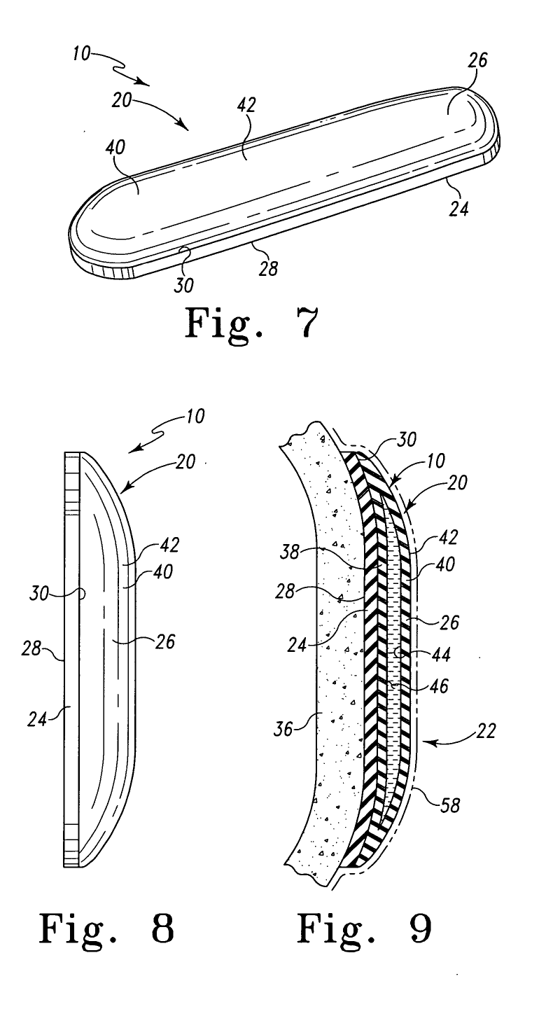

Referring now to FIGS. 1-9, there is shown an inflatable facial implant 10. The implant 10 may be implanted into a patient during reconstructive or cosmetic facial surgery. For example, the implant 10 may be embodied as a malar implant 12 (see FIGS. 1-3) and implanted into the cheek 14 of a patient. As shown in FIGS. 4-6, the implant 10 may be embodied as a chin implant 16 and implanted into the chin 18 of a patient. Moreover, as shown in FIGS. 7-9, the implant 10 may be embodied as a nasolabial fold implant 20, and implanted into the nasiolabial fold 22 of the patient. As such, the implant 10 may be used to correct or augment desirable facial features of the patient. It should be appreciated that although the concepts discussed in relation to FIGS. 1-9 are herein exemplarily described in regard to malar, chin, and nasiolabial fold implant, such concepts may be utilized in conjunction with an implant for use in other reconstructive or cosmetic facial procedures. For example, such concepts may also be utilized in the construction of a meliolabial fold implant, glabellar fold implant, lip augmentation implant, nasal implant, or any other type of facial implant.

The implant 10 has a base 24 with a bladder 26 secured thereto. The base 24 has an inner side 28 and an outer side 30. As shown in FIGS. 3, 6, and 9, at least a portion of the inner side 28 of the base 24 is concave, although configurations other than concave may be used. The inner side 28 of the base 24 is contoured to substantially conform to a subcutaneous anatomical structure of the patient such as soft tissue or bone.

For example, the inner side 28 of the base 24 of the malar implant 12 is contoured to substantially conform to the cheekbone 32 (i.e., the malar/zygomatic bone) of the patient's cheek 14 (see FIG. 3). As such, the malar implant 12 may be provided in a number of different shapes and sizes to accommodate different patient anatomies or desired surgical results. Moreover, the contour of the inner side 28 of the base 24 of the malar implant 12 may be configured to accommodate any soft tissue which may be disposed between the implant 12 and the cheekbone 32.

Similarly, as shown in FIG. 6, the inner side 28 of the base 24 of the chin implant 16 is contoured to substantially conform to the mandible 34 of the patient's chin 18. As with the malar implant 12, the chin implant 16 may be provided in a number of different shapes and sizes to accommodate different patient anatomies or desired surgical results. Moreover, the contour of the inner side 28 of the base 24 of the chin implant 16 may be configured to accommodate any soft tissue which may be disposed between the implant 16 and the mandible 34.

As shown in FIG. 9, the inner side 28 of the base 24 of the nasiolabial fold implant 20 is contoured to substantially conform to the underlying soft tissue 36 of the patient's nasiolabial fold 22. As such, the nasiolabial fold implant 20 may be provided in a number of different shapes and sizes to accommodate different patient anatomies or desired surgical results.

It should be appreciated that the base 24 of other types of facial implants (e.g., a meliolabial fold implant, glabellar fold implant, lip augmentation implant, nasal implant, or any other type of facial implant) may be contoured to likewise conform to a subcutaneous anatomical structure (e.g., soft tissue or bone).

The base 24 may be constructed with any type of rigid or semi-rigid implant grade material. In exemplary embodiments, the base may be constructed from rigid or semi-rigid silicone rubber (e.g., silastic), poly vinyl chloride (PVC), polyurethane, polytetrafluoroethylene (PTFE), or elastomeric hydrogels. Other materials may be used as well.

In the exemplary embodiment described herein, the bladder 26 includes an inner bladder wall 38 and an outer bladder wall 40. The inner bladder wall 38 is secured to the outer side 30 of the base 24. The outer bladder wall 40 defines a contoured outer surface 42 of the implant 10. As such, the outer bladder wall 40 may be configured with structures and features to produce a desired reconstructive or cosmetic effect. For example, the outer bladder wall 40 may be configured with one or more convex surfaces, concave surfaces, lobes, protrusions, extensions, or any other feature to produce an implant 10 with a desired outer topography. As such, a given type of implant 10 may be provided with outer bladder walls having a number of different shapes, sizes, and contours to accommodate different patient anatomies or desired surgical results.

The bladder walls 38, 40 collectively define a fluid chamber 44 into which a fluid 46 may be injected. It should be appreciated that the contour of the outer surface 42 of the implant 10 may be changed by either increasing or decreasing the amount of fluid 46 present in the fluid chamber 44. For example, in the case of an implant 10 used for augmentation of a facial feature, the degree of augmentation may be increased by increasing the amount of fluid in the fluid chamber 44 or decreased by decreasing the amount of fluid in the fluid chamber. The fluid 46 may be any implant grade fluid such as saline, water, or other fluids and gels commonly employed in medical implants.

Moreover, the fluid chamber 44 may be configured to include a number of sub-chambers (not shown) by the use of one or more internal bladder walls (not shown). In such a way, a portion of the implant 10 may be inflated or deflated independently of the other(s).

The bladder 26 may be constructed with any type of flexible or semi-flexible implant grade material. In exemplary embodiments, the bladder 26 may be constructed from flexible or semi-flexible silicone rubber (e.g., silastic), poly vinyl chloride (PVC), polyurethane, polytetrafluoroethylene (PTFE), or elastomeric hydrogels. Other materials may be used as well.

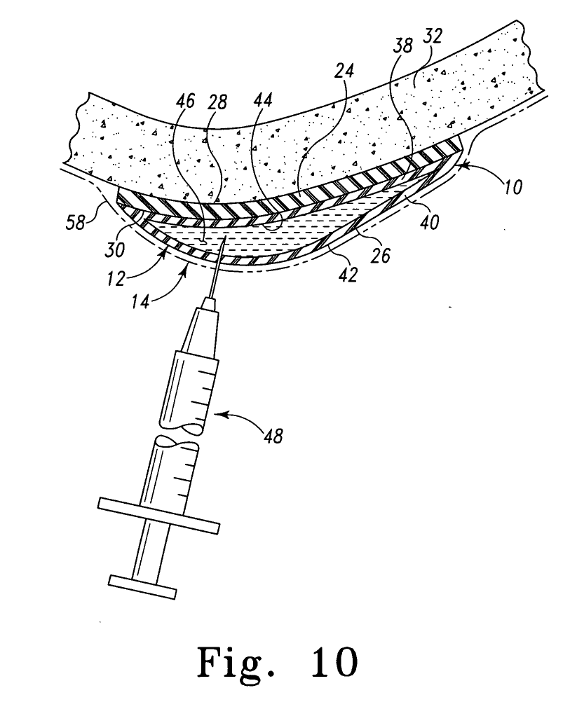

In an exemplary embodiment, the bladder 26 is constructed with a self-sealing polymer such as self-sealing silicone. In such a way, a needle 48, such as a non-coring 20-22 ga. Huber needle, may be used to pierce the bladder 26 to add or remove fluid 46 from the fluid chamber 44 (see FIG. 10). The needle 48 may then be removed without fluid 46 leaking from the bladder 26.

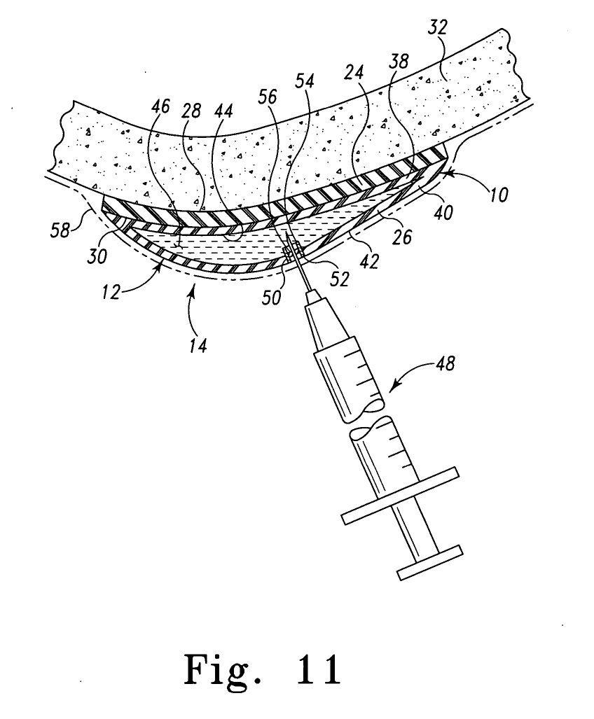

In another exemplary embodiment, a subcutaneous port 50 is positioned in the outer bladder wall 40 (see FIG. 11). The subcutaneous port 50 has an outer end 52 which is disposed relatively flush-mount with the contoured outer surface 42 of the implant 10 (i.e., level with the outer surface 42 or slightly recessed therein). The inner end 54 of the subcutaneous port 50 extends into the fluid chamber 44. The subcutaneous port 50 may be constructed from a number of different materials such as stainless steel, titanium, or various plastics. The subcutaneous port 50 may also have one or more magnetic features to facilitate locating the port 50 with the needle 48. The subcutaneous port 50 also includes a self-sealing septum 56. The septum 56 may be made of a self-sealing polymer such as self-sealing silicone. In such a way, the needle 48 may be used to pierce the septum 56 to gain access into the fluid chamber 44 via the port 50 to add or remove fluid 46 from the fluid chamber 44. The needle 48 may then be removed without fluid 46 leaking from the bladder 26.

Although the bladder 26 is herein exemplarily described as having two bladder walls 38, 40, it should be appreciated that other configurations are also possible. For example, the bladder 26 may be configured without an inner bladder wall 38, with the outer bladder wall 40 being secured to the base 24. In such a case, the fluid chamber 44 is defined by the outer bladder wall 40 and the outer side 30 of the base 24.

In an exemplary surgical method, the implant 10 is implanted as part of a reconstructive or cosmetic surgical procedure. A local or tumescent anesthesia (e.g., in the form of topical anesthetic), with or without sedation, may be used at the commencement of the procedure. Alternatively, the implant may be placed as an adjunctive procedure with a general anesthetic being used, if desired. An incision may then be made in the skin in langerhans lines (e.g., the resting skin tension lines). Thereafter, the associated subcutaneous tissue (collectively designated with reference numeral 58) is dissected or undermined only to encompass the implant in the location of the soft tissue defect in a manner consistent with the implantation of a conventional (i.e., non-inflatable) facial implant of the same type. It should be appreciated that a template of the soft tissue defect may be made preoperatively to determine the size and type of implant for use. Moreover, a measurement device may be used to measure the depth of the defect.

Prior to implantation, an initial quantity of fluid 46 is injected into the fluid chamber 44 of the sterile implant 10. In doing so, the surgeon can create an initial configuration of the size, shape, and contour of the implant 10 by controlling the amount of fluid 46 that is injected. The fluid 46 may be injected through either the self-sealing bladder 26 (if the implant 10 is so designed) or through the port 50 (if the implant 10 is so designed).

The implant 10 is then inserted through the incision and any dissected soft tissue thereunder. The implant 10 is placed in the surrounding soft tissue such that the inner side 28 of the base 24 contacts the appropriate subcutaneous tissue such as bone or the soft tissue surrounding the bone. Once placed, the surgeon may then intra-operatively add or remove fluid 46 from bladder 26, if desired. Thereafter, the incision is closed.

Subsequent to the surgical procedure, the size, shape, and/or contour of the implanted facial implant 10 may be altered by injecting additional fluid 46 into the bladder 26 or removing fluid 46 from the bladder 26. To do so, the needle 48 may be used to percutaneously pierce either the self-sealing outer bladder wall 40 (if the implant 10 is so designed) or the self-sealing septum 56 of the subcutaneous port 50 (if the implant 10 is so designed). It should be appreciated that a magnetic or other type of metallic locator associated with the implant 10 may be used to locate a desired needle insertion location of the implant 10 (e.g., a magnetic feature associated with the percutaneous port 50 may be used to located the port 50). Once the tip of the needle 48 has gained access to the fluid chamber 44, additional fluid 46 may be injected into the bladder 26 or excess fluid 46 drawn from the bladder 26. The needle 48 may then be removed from the bladder 26 and the patient's tissue.

It should be appreciated that such a method allows for adjustment of the implant 10 without the creation of a subsequent incision. In other words, the size, shape, and/or contour of the implant 10 may be altered over time without the need for a revision surgery to replace the implant. Such is particularly useful in regard to facial implants which are initially used in a cosmetic surgery to combat the effects of aging since such an implant can be routinely adjusted as the patient continues to age.

While the concepts of the present disclosure have been illustrated and described in detail in the drawings and foregoing description, such an illustration and description is to be considered as exemplary and not restrictive in character, it being understood that only the illustrative embodiments have been shown and described and that all changes and modifications that come within the spirit of the disclosure are desired to be protected.

There are a plurality of advantages of the present disclosure arising from the various features of the apparatus and methods described herein. It will be noted that alternative embodiments of the apparatus and methods of the present disclosure may not include all of the features described yet still benefit from at least some of the advantages of such features. Those of ordinary skill in the art may readily devise their own implementations of an apparatus and method that incorporate one or more of the features of the present disclosure and fall within the spirit and scope of the present disclosure.

1. An inflatable facial implant, comprising:
a base having a first side and a second side, and
a bladder wall secured to the first side of the base, the bladder wall being more flexible than the base.

2. The facial implant of claim 1, wherein at least a portion of the second side of the base is concave.

3. The facial implant of claim 1, wherein the second side of the base is contoured to substantially conform to a subcutaneous anatomical structure of a human.

4. The facial implant of claim 1, wherein the second side of the base is contoured to substantially conform to a portion of a cheekbone of a human.

5. The facial implant of claim 4, wherein the second side of the base is contoured to substantially conform to a portion of a mandible of a human.

6. The facial implant of claim 1, wherein the second side of the base is contoured to substantially conform to a portion of soft tissue of a human.

7. The facial implant of claim 1, further comprising a fill port positioned in the bladder wall.

8. The facial implant of claim 1, wherein the bladder wall is constructed of a self-sealing polymer.

9. An inflatable facial implant, comprising:
a base having a first side and a second side, the second side of the base being contoured to substantially conform to a subcutaneous anatomical structure of a human face, and
a bladder wall secured to the first side of the base.

10. The facial implant of claim 9, wherein at least a portion of the second side of the base is concave.

11. The facial implant of claim 9, wherein the bladder wall defines a chamber for receiving a fluid.

12. The facial implant of claim 9, wherein the second side of the base is contoured to substantially conform to a portion of a cheekbone of a human.

13. The facial implant of claim 9, wherein the second side of the base is contoured to substantially conform to a portion of a mandible of a human.

14. The facial implant of claim 9, wherein the second side of the base is contoured to substantially conform to a portion of soft tissue of a human.

15. The facial implant of claim 9, further comprising a fill port positioned in the bladder wall.

16. The facial implant of claim 9, wherein the bladder wall is constructed of a self-sealing polymer.

17. A surgical method, comprising the steps of:
injecting an initial quantity of fluid into a bladder of a facial implant, the bladder of the facial implant being secured to a base which is less flexible than the bladder,
implanting the facial implant into a patient such that a base of the facial implant contacts subcutaneous tissue, and
percutaneously injecting an additional quantity of fluid into the bladder of the facial implant subsequent to the implanting step.

18. The method of claim 17, wherein:
the bladder is constructed of a self-sealing polymer, and
the percutaneously injecting step comprises piercing the bladder with a needle and injecting the additional quantity of fluid into the bladder via the needle.

19. The method of claim 17, wherein:
the facial implant comprises a subcutaneous port having a self-sealing septum, and the percutaneously injecting step comprises piercing the septum with a
needle and injecting the additional quantity of fluid into the bladder via the needle.

20. The method of claim 17, further comprising the step of percutaneously removing a quantity of fluid in the bladder of the facial implant subsequent to the implanting step.

21. The method of claim 20, wherein:
the bladder is constructed of a self-sealing polymer, and
the percutaneously removing step comprises piercing the bladder with a needle and removing the quantity of fluid from the bladder via the needle.

22. The method of claim 20, wherein:
the facial implant comprises a subcutaneous port having a self-sealing septum, and
the percutaneously removing step comprises piercing the septum with a needle and removing the quantity of fluid from the bladder via the needle.

23. The method of claim 17, wherein:
the facial implant comprises a malar implant, and
the implanting step comprises implanting the malar implant into the cheek of the patient.

24. The method of claim 17, wherein:
the facial implant comprises a chin implant, and
the implanting step comprises implanting the chin implant into the chin of the patient.

25. The method of claim 17, wherein:
the facial implant comprises a nasolabial implant, and the implanting step comprises implanting the nasolabial implant into the nasolabial fold of the patient.

26. An inflatable facial implant, comprising:
an inflatable bladder having a convex outer wall, and
a subcutaneous port positioned in the convex outer wall.

27. The inflatable facial implant of claim 26, wherein the subcutaneous port comprises a self-sealing septum.

28. The facial implant of claim 26, further comprising a base having a first side thereof secured to the inflatable bladder, wherein at least a portion of a second side of the base is concave.

29. The facial implant of claim 28, wherein the second side of the base is contoured to substantially conform to a subcutaneous anatomical structure of a human.

30. The facial implant of claim 28, wherein the second side of the base is contoured to substantially conform to a portion of a cheekbone of a human.

31. The facial implant of claim 28, wherein the second side of the base is contoured to substantially conform to a portion of a mandible of a human.

32. The facial implant of claim 28, wherein the second side of the base is contoured to substantially conform to a portion of soft tissue of a human.
